(19) United States
(12) Patent Application Publication
(10) Pub. No.: US 2023/0285074 A1
(43) Pub. Date: Sep. 14, 2023

(54) MEDICAL DEVICES AND METHODS FOR CARRYING OUT A MEDICAL PROCEDURE

(71) Applicant: Boston Scientific Medical Device Limited, Galway (IE)

(72) Inventors: Charlene Leung, Scarborough (CA); Lauren Koon, Mississauga (CA); Eduardo Moriyama, Toronto (CA); Gareth Davies, Toronto (CA)

(21) Appl. No.: 18/181,935

(22) Filed: Mar. 10, 2023

Related U.S. Application Data
(60) Provisional application No. 63/318,856, filed on Mar. 11, 2022.

(51) Int. Cl. A61B 18/14

(52) U.S. Cl. CPC A61B 18/1492; A61B 2017/00247

(57) ABSTRACT

A method for carrying out a medical procedure includes advancing a medical device towards a puncture site in a patient's body and positioning a radiofrequency puncture electrode of the medical device adjacent the puncture site; delivering radiofrequency energy from the radiofrequency puncture electrode to create a puncture in the puncture site; advancing the medical device through the puncture to position an auxiliary electrode of the medical device adjacent a target site in the patient's body; and using the auxiliary electrode for diagnosis, mapping, and/or treatment at the target site.

### CROSS REFERENCE TO RELATED APPLICATION

This application claims priority to U.S. Provisional Patent Application No. 63/318,856, filed Mar. 11, 2022.

### FIELD

This document relates to medical devices and medical procedures. More specifically, this document relates to intravascular and intracardiac medical procedures, and related devices.

### SUMMARY

The following summary is intended to introduce the reader to various aspects of the detailed description, but not to define or delimit any invention.

Methods for carrying out medical procedures are disclosed. According to some aspects, a method for carrying out a medical procedure includes: a. advancing a medical device towards a puncture site in a patient's body and positioning a radiofrequency puncture electrode of the medical device adjacent the puncture site; b. delivering radiofrequency energy from the radiofrequency puncture electrode to create a puncture in the puncture site; c. advancing the medical device through the puncture to position an auxiliary electrode of the medical device at a target site in the patient's body; and d. using the auxiliary electrode for diagnosis, mapping, and/or treatment at the target site.

In some examples, the puncture site is a fossa ovalis of the patient's heart, and the target site is a structure of a left side of the patient's heart.

In some examples, step c. includes using the auxiliary electrode for pulmonary vein isolation.

In some examples, the target site is the ventricular endocardium.

In some examples, step d. includes using the auxiliary electrode for electroanatomic mapping of the target site. In some examples, step d. includes using the auxiliary electrode to ablate the target site. In some examples, step d. includes using the auxiliary electrode to collect electrical signals from the target site. In some examples, step d. includes using the auxiliary electrode to pace the target site. In alternative embodiments, the medical device is operable in a bipolar manner, in which case the auxiliary electrode may be used as a grounding electrode. A bipolar device may be further beneficial in that the energy delivered via the medical device would remain concentrated around the two electrodes, substantially limiting the undesirable flow of current through structures such as the heart.

In alternative aspects, a method for carrying out a medical procedure includes: a. advancing a medical device towards a puncture site in a patient's body and positioning a radiofrequency puncture electrode of the medical device adjacent the puncture site; b. positioning an auxiliary electrode of the medical device at a puncture site in the patient's body and using the auxiliary electrode for diagnosis, and/or mapping at a target site; c. delivering radiofrequency energy from the radiofrequency puncture electrode to create a puncture in the puncture site; and d. advancing the medical device through the puncture to reach the target site; and f. optionally, positioning the auxiliary electrode of the medical device at the target site in the patient's body.

In some examples, steps b. and f. include using the auxiliary electrode for pulmonary vein isolation.

In some examples, steps b. and f. include using the auxiliary electrode for electroanatomic mapping of the target site. In some examples, step f. includes using the auxiliary electrode to ablate the target site. In some examples, steps b. and f. include using the auxiliary electrode to collect electrical signals from the target site. In some examples, steps b. and f. include using the auxiliary electrode to pace the target site.

In some examples, the auxiliary electrode is positioned in a shape-changing section of the medical device, and the method further includes changing the shape of the shape-changing section to anchor the shape-changing section in a vessel.

In some examples, the target site includes a wall of the vessel, and changing the shape of the shape-changing section positions the auxiliary electrode in contact with the target site.

In some examples, the method further includes using the medical device as a rail to advance a secondary device towards the target site.

In some examples, step d. includes using the auxiliary electrode to perform a treatment at the target site, and then using the auxiliary electrode to confirm the treatment.

Medical devices are also disclosed. According to some aspects, a medical device includes an elongate member having a distal portion defining a distal end, and a proximal portion defining a proximal end. A radiofrequency puncture electrode is at the distal end, and a first electrical connector extends proximally from the radiofrequency puncture electrode towards the proximal end, for electrically connecting the radiofrequency puncture electrode to a radiofrequency generator. At least a first auxiliary electrode is in the distal portion, positioned proximally of the radiofrequency puncture electrode. A second electrical connector extends proximally from the auxiliary electrode towards the proximal end, for electrically connecting the auxiliary electrode to a diagnostic system, a mapping system, and/or a treatment system.

In some examples, the first auxiliary electrode is one of a plurality of auxiliary electrodes that are longitudinally spaced apart in the distal portion.

In some examples, the distal portion includes a shape-changing section, and the first auxiliary electrode is in the shape-changing section.

In some examples, the shape-changing section is changeable from a generally straight shape to a loop shape, a pigtail shape, a balloon shape, a basket shape, a J-shape, a semi-circular shape, or a combination thereof.

In some examples, the elongate member is relatively stiff proximal of the shape-changing section.

In some examples, the medical device is a guidewire, and the elongate member has an outer diameter of between about 0.014 inches to about 0.060 inches, more preferably between about 0.020 inches to about 0.040 inches.

In some examples, the medical device is a microcatheter and the elongate member has an outer diameter of between about 1.5F (0.02 inches) to about 6F (0.079 inches), more preferably between 2F (0.026 inches) to 3F (0.039 inches).

In some examples, the elongate member comprises a central mandrel and an outer liner.

### BRIEF DESCRIPTION OF THE DRAWINGS

The accompanying drawings are for illustrating examples of articles, methods, and apparatuses of the present disclosure and are not intended to be limiting. In the drawings:

FIG. 1 is a perspective view of an example medical system, including a radiofrequency generator, an auxiliary system, a sheath, a dilator, and a medical device;

FIG. 2 is a side view of the medical device of FIG. 1;

FIG. 3 is a longitudinal cross-section taken through a portion of the medical device of FIG. 2;

FIG. 4 is a partial side view of another example medical device;

FIG. 5 is a partial side view of another example medical device;

FIG. 6 is a partial side view of another example medical device;

FIG. 7 is a partial side view of another example medical device;

FIG. 8 is a partial side view of another example medical device;

FIG. 9 is a partial side view of another example medical device;

FIG. 10 shows a step of a method for carrying out a medical procedure, using the sheath and dilator of the system of FIG. 1, and the medical device of FIG. 5;

FIG. 11 shows a subsequent step of the method of FIG. 10;

FIG. 12 shows a subsequent step of the method of FIG. 11;

FIG. 13 shows a subsequent step of the method of FIG. 12;

FIG. 14 shows a subsequent step of the method of FIG. 13;

FIG. 15 shows a subsequent step of the method of FIG. 14;

FIG. 16 shows a step of another method for carrying out a medical procedure, using the sheath of FIG. 1 and the medical device of FIG. 8.

### DETAILED DESCRIPTION

Various apparatuses or processes or compositions will be described below to provide an example of an embodiment of the claimed subject matter. No example described below limits any claim and any claim may cover processes or apparatuses or compositions that differ from those described below. The claims are not limited to apparatuses or processes or compositions having all of the features of any one apparatus or process, or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Generally disclosed herein are methods for carrying out medical procedures, and related medical devices. More specifically, disclosed herein are medical procedures in which a first anatomical structure (also referred to herein as a “puncture site”) is punctured to gain access to a second anatomical structure (also referred to herein as a “target site”), and then a procedure (e.g., diagnosis, mapping, and/or treatment) is carried out in relation to the target site. In an alternative example, a procedure (e.g., diagnosis and/or mapping) is carried out before the first anatomical structure is punctured. In the methods disclosed herein, a single medical device is used to both puncture the puncture site and to carry out the procedure in relation to the target site. For example, the medical device may be in the form of a guidewire or microcatheter, and may include a radiofrequency electrode at its distal end, and one or more auxiliary electrodes that are positioned proximally of the radiofrequency electrode. The medical device may be advanced towards a puncture site in a patient's body and the radiofrequency puncture electrode may be positioned adjacent the puncture site. Radiofrequency energy may then be delivered from the radiofrequency puncture electrode to create a puncture in the puncture site. The medical device may then be advanced through the puncture to position the auxiliary electrode(s) of the medical device adjacent a target site, and the auxiliary electrode(s) may then be used for diagnosis, mapping, and/or treatment at the target site. Alternatively, the medical device may be advanced towards a puncture site in a patient's body and the radiofrequency puncture electrode may be positioned adjacent the puncture site and the auxiliary electrode(s) may then be used for diagnosis and/or mapping of the target site. Radiofrequency energy may then be delivered from the radiofrequency puncture electrode to create a puncture in the puncture site, and then advancing the medical device through the puncture to position the auxiliary electrode(s) of the medical device adjacent a target site, and, using the auxiliary electrode(s) for diagnosis, mapping, and/or treatment at the target site.

In some examples, the puncture site may be the fossa ovalis of a patient's heart, and the target site may be a structure of the left side of the patient's heart. For example, the methods may involve pulmonary vein isolation. In such examples, the radiofrequency puncture electrode may be used to puncture the fossa ovalis of the heart, to gain access to the left side of the heart. The auxiliary electrode(s) may then be positioned in or adjacent a pulmonary vein, and may be used to ablate the wall of the pulmonary vein. The auxiliary electrode(s) may then further be used to assess whether the pulmonary vein isolation was successful (i.e. i.e. the auxiliary electrode(s) may collect electrical signals).

In other examples, the methods may involve ventricular endocardial ablation. In such examples, the radiofrequency puncture electrode may be used to puncture the fossa ovalis of the heart, to gain access to the left side of the heart. The auxiliary electrode(s) may then be positioned in the left ventricle, and may be used collect an ECG signal, a mapping signal, and/or other diagnostic information. The auxiliary electrode(s) may then be used to ablate endocardial tissue. The auxiliary electrode(s) may then again be used to collect electrical signals, to assess whether the procedure was successful.

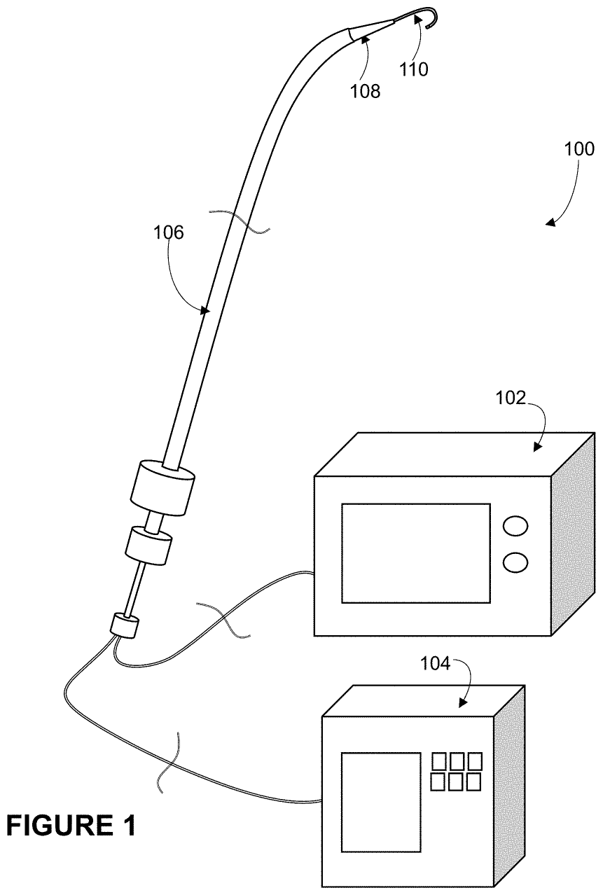

Referring now to FIG. 1, an example medical system 100 is shown. The system generally includes a radiofrequency generator 102, an auxiliary system 104, a sheath 106, a dilator 108, and a medical device 110. The medical device 110 is electrically connected to the radiofrequency generator 102 and the auxiliary system 104, and is insertable through the dilator 108 and the sheath 106, towards a target site in a patient's body.

The radiofrequency generator 102 may be any suitable generator that can deliver radiofrequency energy to the medical device 110, to allow for the medical device 110 to puncture tissue. One such generator is sold by Baylis Medical Company (Montreal, Canada) under the name RFP-100A RF Puncture Generator.

The auxiliary system 104 may be, for example, a diagnostic system (e.g. an electrocardiogram system, such as CardioLab™ sold by GE Healthcare), a mapping system (e.g. an electroanatomical mapping system, such as RHYTHMIA HDx™ by Boston Scientific, CARTO® by Biosense Webster, and EnSite™ by Abbot), and/or a treatment system (e.g. a radiofrequency ablation generator or a pacing system, such as RFP-100A RF Puncture Generator by Baylis Medical Company, CardioLab™, FARASTAR™, and SMARTABLATE®.

In the example shown, the radiofrequency generator 102 and the auxiliary system 104 are shown as two separate sub-systems; however, they may be integrated into a single system. Furthermore, in some examples, more than one auxiliary system may be provided (e.g., a diagnostic system, a mapping system, and a treatment system), which may or may not be integrated into a single overall system. If more than one auxiliary system is provided, each auxiliary system may be connected to the medical device 110 at the same time, or the auxiliary systems may be connected to the medical device 110 in turn.

The sheath 106 may be any suitable sheath that can provide a conduit for the medical device 110, and the dilator 108 may be any suitable dilator that can dilate a puncture created by the medical device 110. Examples include the TorFlex™ Transseptal Guiding Sheath, SureFlex® Steerable Guiding Sheath, VersaCross® Large Access Transseptal Dilator, regular VersaCross® Transseptal Dilator, and ExpanSure® Large Access Transseptal Dilator, each of which is sold by Baylis Medical Company (Montreal, Canada).

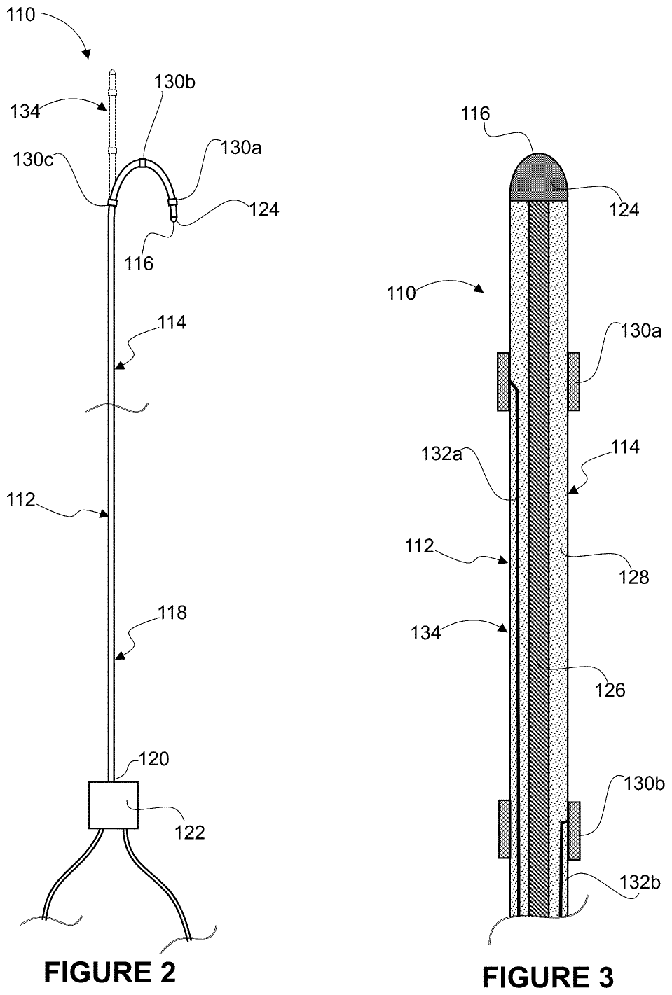

Referring now to FIG. 2, the medical device 110 is shown in greater detail. In the example shown, the medical device 110 includes an elongate member 112, which has a distal portion 114 defining a distal end 116 and a proximal portion 118 defining a proximal end 120. The length between the distal end 116 and the proximal end 120 may be, for example, between about 180 cm to about 230 cm. In an embodiment, about 180 cm, or about 187 cm. A hub 122 is joined the proximal end 120, and a radiofrequency puncture electrode 124 is at the distal end 116. As shown in FIG. 3, the elongate member 112 includes a central mandrel 126 (such as a solid body or rod, or a hollow body or tube), and a liner 128 on the central mandrel. The central mandrel 126 serves as an electrical connector for the radiofrequency puncture electrode 124, and extends proximally from the radiofrequency puncture electrode 124 towards the proximal end 120 (not shown in FIG. 3), for electrically connecting the radiofrequency puncture electrode 124 to the RF generator 102 (not shown in FIGS. 2 and 3) via the hub 122 (not shown in FIG. 3). The liner 128 electrically insulates the mandrel 126 (e.g., the liner 128 may be a polytetrafluoroethylene (PTFE) liner). The mandrel 126 further provides stiffness to the elongate member 112. In the example shown, the mandrel 126 is of a constant diameter along its length; however, in alternative examples, the mandrel may increase in diameter as it extends towards the proximal end, in order to provide increased stiffness. In further alternative examples, the mandrel may include a stiffening member that is inserted into a lumen thereof, in order to provide increased stiffness.

Referring still to FIGS. 2 and 3, in the example shown, the elongate member 112 further includes a plurality of auxiliary electrodes, namely a first auxiliary electrode 130a, a second auxiliary electrode 130b, and a third auxiliary electrode 130c (shown only in FIG. 2). The auxiliary electrodes 130a-c are positioned in the distal portion 114, proximally of the radiofrequency puncture electrode 124, and are longitudinally spaced apart. In the example shown, the auxiliary electrodes 130a-c are in the form of ring electrodes that are received on the liner 128. As shown in FIG. 3, the elongate member 112 further includes a respective electrical connector for each auxiliary electrode (only the electrical connector 132a for the first auxiliary electrode 130a and the electrical connector 132b for the second auxiliary electrode 130b are shown). In an example, said electrical connector may have multiple pathways for each device/system for the electrodes being used. Alternatively, the electrical connector may be OTW or OTG cable-cable connector. The electrical connectors 132a-b are in the form of insulated wires that are embedded in the liner 128, and each electrical connector 132a-b extends proximally from each respective auxiliary electrode 130a-b towards the proximal end 120, for electrically connecting the respective auxiliary electrode 130a-b to the auxiliary system 104 via the hub 122. In further alternative examples, the mandrel may be a long metal tube (e.g., a hypotube) with an insulated outside diameter (OD) comprising an insulated wire extending longitudinally therethrough. The insulated wire serves as an electrical connector for the radiofrequency puncture electrode 124, and extends proximally from the radiofrequency puncture electrode 124 towards the proximal end 120, for electrically connecting the radiofrequency puncture electrode 124 to the RF generator 102 via the hub 122. The hypotube may include stainless steel, Nitinol, or both. Further, the hypotube may be made of metal, plastic, polymers, or any combination thereof and may be laser cut to modify or vary its flexibility or stiffness (e.g., laser cut stainless steel). In alternative examples, the hub 122 coupled to the proximal end 120 of the elongated member 112 is a separate member and removable from the wire or microcatheter (i.e., a detachable cable can connect the elongated member and the generator), thereby allowing other devices to be slid over the elongated member. In alternative examples, the hub is rotatable with respect to the elongated member while maintaining communication with the radiofrequency generator 102, an auxiliary system 104. In such procedures, the hub may be locked or coupled to the elongated member using a coupling mechanism during a portion of the procedure, preventing undesired disengagement and/or rotation of the hub from the elongated member.

In the example shown, the medical device 110 includes 3 auxiliary electrodes 130a-c; however, in alternative examples, the medical device may include another number of auxiliary electrodes (i.e., at least one auxiliary electrode, and preferably a plurality of auxiliary electrodes, such as 1 or 2 or 4 or up to 8 auxiliary electrodes).

Referring still to FIG. 2, in the example shown, the distal portion 114 of the elongate member 112 includes a shape-changing section 134. The shape changing section 134 is changeable between a generally straight shape (shown in dotted line in FIG. 2, and also shown in FIG. 3) and a J-shape (shown in solid line in FIG. 2). In the example shown, the elongate member 112 is biased towards the J-shape, but may assume the generally straight shape on the application of force (e.g. when confined within the sheath 106 and/or dilator 108). For example, the shape-changing section 134 may include a shape-memory material. In alternative examples (not shown), one or more actuation mechanisms may be provided, for selectively moving the elongate member between the generally straight shape and the J-shape.

The shape changing section 134 may generally serve two purposes: Firstly, as the medical device 110 is advanced out of the sheath 106 and/or dilator 108 to puncture tissue (e.g. the fossa ovalis), the elongate member 112 will assume the J-shape. This causes the radiofrequency puncture electrode 124 to turn away from additional tissue that is in front of the radiofrequency puncture electrode 124 (e.g. the wall of the left atrium), to prevent inadvertent damage to the additional tissue. Secondly, as can be seen in FIG. 2, the auxiliary electrodes 130a-c are in the shape-changing section. As will be described in greater detail below, the shape-changing section 134 can allow for the auxiliary electrodes 130a-c to be anchored in and/or positioned against a target site (e.g. a pulmonary vein), to facilitate use of the auxiliary electrodes 130a-c.

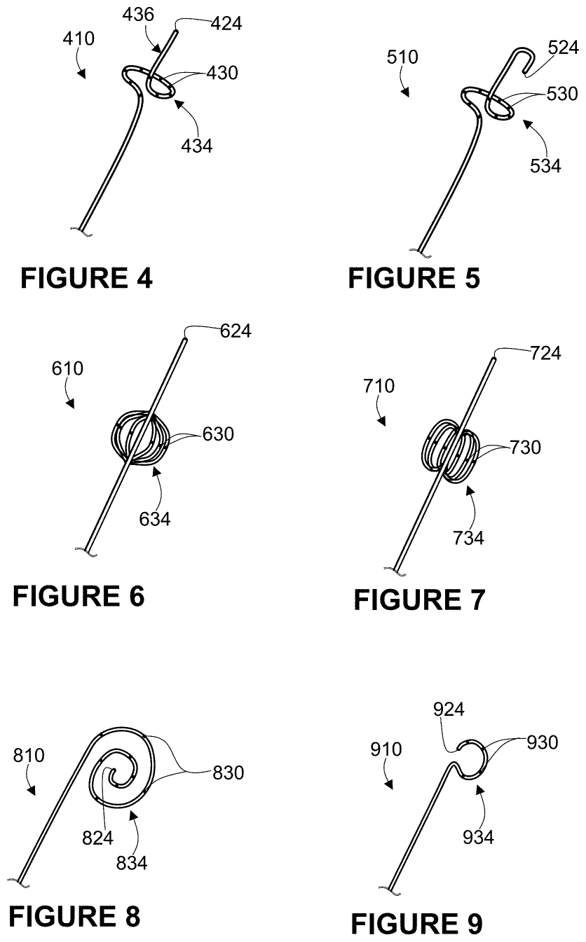

In the example shown in FIGS. 2 and 3, the shape-changing section 134 is changeable between a generally straight shape and a J-shape. As shown in FIGS. 4 to 9, in alternative examples, the shape-changing section can be changeable between a generally straight shape and a variety of other shapes.

For example, referring to FIG. 4, a medical device 410 is shown that includes a shape-changing section 434 that is changeable between a generally straight shape (not shown) and a loop shape. Similarly to the medical device 110, in the medical device 410, the auxiliary electrodes 430 (only two of which are labelled) are in the shape-changing section 434 and the radiofrequency puncture electrode 424 is at the distal end.

For further example, referring to FIG. 5, a medical device 510 is shown that includes a shape-changing section 534 that is changeable between a generally straight shape (not shown) and a combination of a loop shape and a J-shape. Similarly to the medical device 110, in the medical device 510, the auxiliary electrodes 530 (only two of which are labelled) are in the shape-changing section 534 and the radiofrequency puncture electrode 524 is at the distal end.

For further example, as shown in FIG. 6, a medical device 610 is shown that includes a shape-changing section 634 that is changeable between a generally straight shape (not shown) and a balloon shape. Similarly, to the medical device 110, in the medical device 610, the auxiliary electrodes 630 (only two of which are labelled) are in the shape-changing section 634 and the radiofrequency puncture electrode 624 is at the distal end.

For further example, as shown in FIG. 7, a medical device 710 is shown that includes a shape-changing section 734 that is changeable between a generally straight shape (not shown) and a basket shape. Similarly, to the medical device 110, in the medical device 710, the auxiliary electrodes 730 (only two of which are labelled) are in the shape-changing section 734 and the radiofrequency puncture electrode 724 is at the distal end.

For further example, as shown in FIG. 8, a medical device 810 is shown that includes a shape-changing section 834 that is changeable between a generally straight shape (not shown) and a pigtail shape. Similarly, to the medical device 110, in the medical device 810, the auxiliary electrodes 830 (only two of which are labelled) are in the shape-changing section 834 and the radiofrequency puncture electrode 824 is at the distal end.

For further example, as shown in FIG. 9, a medical device 910 is shown that includes a shape-changing section 934 that is changeable between a generally straight shape (not shown) and a semi-circular shape. Similarly, to the medical device 110, in the medical device 910, the auxiliary electrodes 930 (only two of which are labelled) are in the shape-changing section 934 and the radiofrequency puncture electrode 924 is at the distal end.

In some examples, such as in the medical device 410 of FIG. 4, the shape-changing section 434 is spaced from the radiofrequency puncture electrode 424, with an intermediate section 436 therebetween. The intermediate section 436 may have a length of, for example, about 2 cm. In the example shown, the section of the medical device 410 that is immediately proximal to the shape-changing section 434 is co-linear with the intermediate section 436, to facilitate navigation.

In some examples, the elongate member can be relatively stiff proximal of the shape changing section. For example, the mandrel may increase in diameter proximal of the shape changing section, or may include a stiffening member proximal of the shape-changing section.

In some examples, in which the medical device is relatively stiff and has a relatively small outer diameter, the medical device may be considered as or referred to as a guidewire. In alternative examples, the medical device may have a larger outer diameter and/or may be relatively non-stiff. In some such examples, the medical device may be considered as or referred to as a microcatheter. For example, the elongate member may in some cases have a diameter of between about 0.014 inches to about 0.060 inches. In embodiments, the elongate member has a diameter between about 0.020 inches to about 0.040 inches. In some embodiments, the elongate member has a diameter of about 0.035 inches and is relatively stiff, and may thus be considered as a guidewire. For further example, the elongate member may in some cases have a diameter of between about 1.5F (0.02 inches) to about 6F (0.079 inches). In some embodiments, the elongate member has a diameter of between about 2F (0.026 inches) to about 3F (0.039 inches) and be relatively non-stiff, and may thus be considered as a microcatheter.

In the examples described above, the medical device includes a radiofrequency puncture electrode, which delivers radiofrequency energy to puncture tissue. In alternative examples, the elongate member may include a sharp tip, and may use mechanical force to puncture tissue.

In any of the above examples, the elongate member may include one or more radiopaque markers and/or one or more echogenic markers. For example, the distal portion may include a tungsten coil.

In any of the above examples, the elongate member may be steerable.

In any of the above examples, the elongate member may include a lumen.

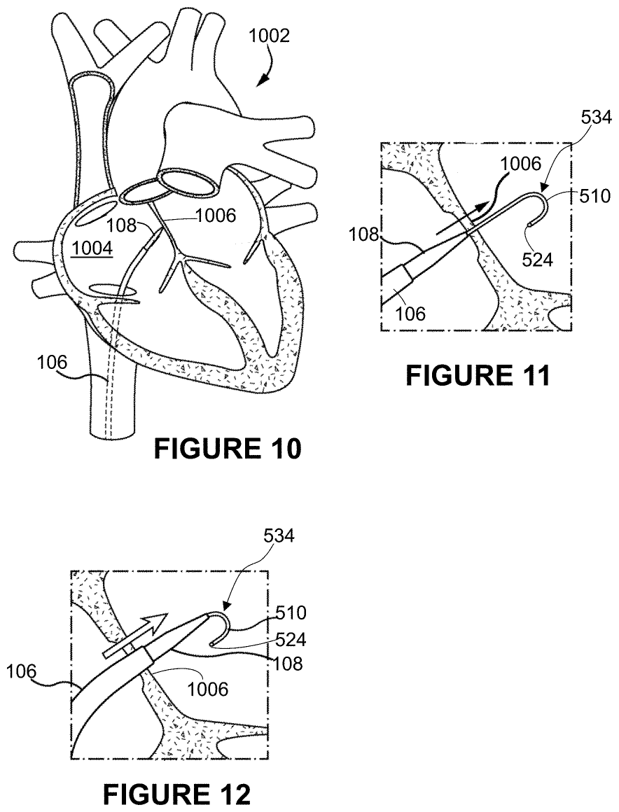

Referring now to FIGS. 10 to 15, a first example method for carrying out a medical procedure will be described. Particularly, a method for pulmonary vein isolation will be described. The method will be described with reference to the medical system 100 and the medical device 500 of FIG. 5; however, the method may be carried out with other systems and devices, and the devices and systems are not limited to use according to the described method.

Referring first to FIG. 10, with the medical device 510 (not visible in FIG. 10), such as guidewire or microcatheter, protrudes from the dilator 108 to enter the vasculature or heart and then the dilator 108 and the sheath 106 can be advanced via the femoral vein towards the heart 1002, and positioned in the right atrium 1004, so that the radiofrequency puncture electrode 524 (not visible in FIG. 10) of the medical device 510 is positioned adjacent the puncture site—i.e. the fossa ovalis 1006—and the auxiliary electrode(s) may then be used for diagnosis, mapping, and/or treatment of the target site prior to puncturing the puncture site. In alternative examples, the medical device 510 may be advanced towards the heart 1002 from another site—e.g., using a superior approach.

Referring to FIG. 11, radiofrequency energy can then be delivered from the radiofrequency generator (not shown) to the radiofrequency puncture electrode 524, and from the radiofrequency puncture electrode 524 to the puncture site, to create a puncture in the fossa ovalis 1006. The radiofrequency energy can be delivered in a continuous signal or a pulsed signal. The medical device 510 can be advanced through the puncture as the energy is delivered, to position the radiofrequency puncture electrode 524 in the right atrium. As the shape-changing section 534 is advanced out of the sheath 106 and dilator 108 to the extent shown in FIG. 11, the shape-changing section 534 will take on a J-shape. As shown in FIG. 12, the dilator 108 can then be advanced through the puncture, to dilate the puncture, and the sheath 106 can then be advanced through the puncture. The dilator 108 can then be withdrawn.

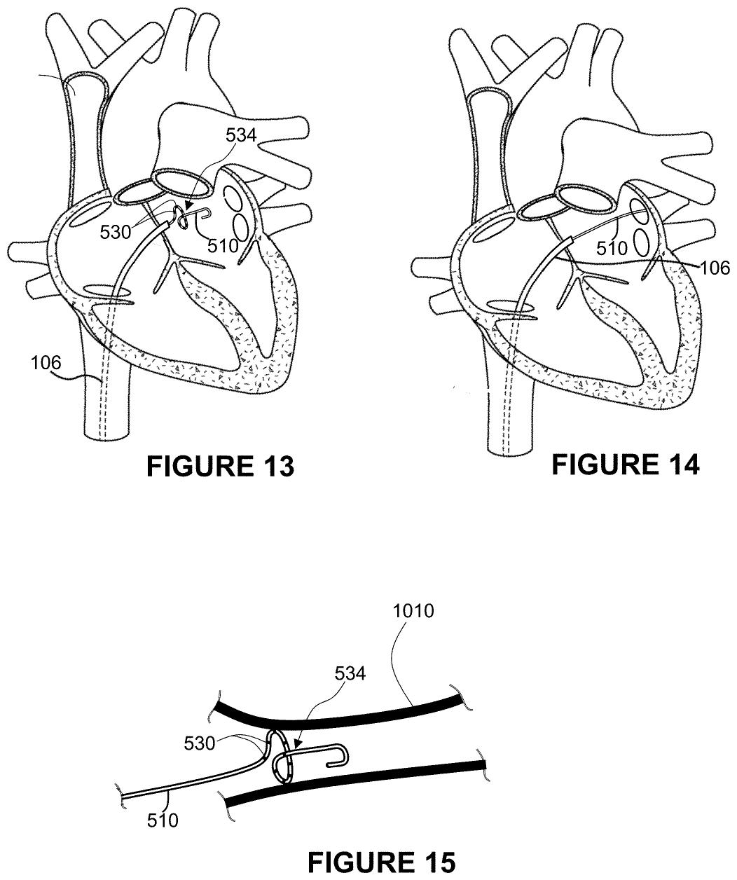

Referring to FIGS. 13 and 14, the medical device 510 can then be further advanced through the puncture, to position the auxiliary electrodes 530 (only two of which are labelled) proud of the sheath 106. As the medical device is further advanced, the shape-changing section 534 will take on a loop-shape, as shown in FIG. 13. The medical device 510 can continue to be advanced until the auxiliary electrodes 530 are adjacent the target site—i.e. a wall 1008 of a pulmonary vein, as shown in FIGS. 14 and 15. More specifically, the medical device 510 can be inserted into the left upper pulmonary vein, so that the loop portion of the shape-changing section 534 anchors in the pulmonary vein, with the auxiliary electrodes 530 in contact with the wall 1010 of the pulmonary vein. The auxiliary electrodes 530 can then be used for diagnosis, mapping, and/or treatment at the target site. For example, prior to treatment, electrical signals can be collected by the auxiliary electrodes 530 and sent to the auxiliary system 104 (not shown), for diagnostic purposes. Energy can then be delivered to the auxiliary electrodes 530 from the auxiliary system 104, to ablate tissue, for the purposes of treatment (e.g. radiofrequency ablation or pulse field ablation). Following ablation, electrical signals can again be collected by the auxiliary electrodes 530 and sent to the auxiliary system 104, to confirm successful ablation. For example, the directionality of electrical signal propagation may be determined, to assess whether the procedure was successful.

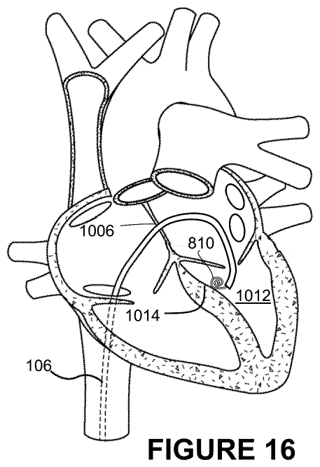

Referring now to FIG. 16, another example method for carrying out a medical procedure will be described. Particularly, a method for ventricular endocardial ablation will be described. The method will be described with reference to the medical system 100 and medical device 810 described above; however, the method may be carried out with other devices, and the devices and systems are not limited to use according to he described method.

The method may begin in a similar fashion to the above-described method for pulmonary vein isolation. That is, FIGS. 10 to 12 are generally applicable to the method for ventricular endocardial ablation, and for brevity, the description thereof is not repeated. Referring to FIG. 16, after the fossa ovalis 1006 has been punctured and the sheath 106 has been advanced through the puncture, the sheath 106 and medical device 810 may be steered towards the left ventricle 1012 and the shape-changing section 834 (labelled in FIG. 8) of medical device 810 may be advanced from the sheath 106, so that it takes on a pigtail shape and so that the auxiliary electrodes 830 (visible in FIG. 8) are in contact with the ventricular endocardium 1014. The auxiliary electrodes 834 can then be used for diagnosis, mapping, and/or treatment at the target site. For example, prior to treatment, an ECG signal, a voltage mapping signal, and/or other signals can be collected by the auxiliary electrodes 830 and sent to the auxiliary system 104, for diagnostic purposes. Energy can then be delivered to the auxiliary electrodes 830 from the auxiliary system 104, for the purposes of treatment. For example, energy can be delivered for ablation (e.g. radiofrequency ablation or pulse field ablation) or for pacing. Following treatment, electrical signals can again be collected by the auxiliary electrodes 830 and sent to the auxiliary system 104, to confirm successful treatment.

In any of the above examples, all of the auxiliary electrodes may be used at a given time (e.g. energy may be delivered to all of the auxiliary electrodes to ablate a large section of tissue), or one or a subset of the auxiliary electrodes may be used at a given time (e.g. energy may be delivered to a single one of the auxiliary electrodes to ablate a smaller section of tissue).

In any of the above examples, after initial use of the auxiliary electrodes, the medical device can be repositioned, and the auxiliary electrodes can be used again.

In any of the above examples, visualization techniques may be used to determine or confirm the position of the distal portion of the medical device. For example, fluoroscopy (e.g. to visualize radiopaque markers on the medical device), computerized tomography, transesophageal echocardiography (to visualize echogenic markers on the medical device) or angiography (to determine the orientation and/or position of the distal portion) may be used.

In any of the above examples, a secondary device, such as a cryoballoon for cryoablation or another ablation device (such as pulsed field ablation (PFA) catheter), can be delivered over the medical device towards the target site, with the medical device serving as a rail.

In any of the above examples, the medical device may be advanced through a supporting device other than a sheath and a dilator. For example, the medical device may be advanced through a microcatheter or coronary catheter. In such examples, the microcatheter be used as an insulator that can reduce influence on electric fields around the auxiliary electrodes.

In any of the above examples, a supporting device such as a stylet may be inserted to a lumen of the elongate member, to facilitate tenting and puncture of the puncture site.

In alternative examples, another area within the patient's body may be an additional or alternative target site, for example the superior vena cava (SVC), the right ventricle, cardiac isthmuses (e.g., mitral isthmus, cavotricuspid isthmus), posterior wall of the left atrium or roofline, the His bundle, the atrioventricular node, the great cardiac vein, the coronary sinus, and/or the anterior interventricular vein (with septal branches).

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A method for carrying out a medical procedure, comprising:
advancing a medical device towards a puncture site in a patient's body and positioning a radiofrequency puncture electrode of the medical device adjacent the puncture site;
delivering radiofrequency energy from the radiofrequency puncture electrode to create a puncture in the puncture site;
advancing the medical device through the puncture to position an auxiliary electrode of the medical device at a target site in the patient's body; and
using the auxiliary electrode for diagnosis, mapping, or treatment at the target site.

2. The method of claim 1, wherein the puncture site is a fossa ovalis of the patient's heart, and the target site is a structure of a left side of the patient's heart.

3. The method of claim 2, wherein advancing the medical device through the puncture comprises using the auxiliary electrode for pulmonary vein isolation.

4. The method of claim 2, wherein the target site is the ventricular endocardium.

5. The method of claim 1, wherein using the auxiliary electrode for diagnosis, mapping, or treatment at the target site comprises using the auxiliary electrode for electroanatomic mapping of the target site.

6. The method of claim 1, wherein using the auxiliary electrode for diagnosis, mapping, or treatment at the target site comprises using the auxiliary electrode to ablate the target site.

7. The method of claim 1, wherein using the auxiliary electrode for diagnosis, mapping, or treatment at the target site comprises using the auxiliary electrode to collect electrical signals from the target site.

8. The method of claim 1, wherein using the auxiliary electrode for diagnosis, mapping, or treatment at the target site comprises using the auxiliary electrode to pace the target site.

9. The method of claim 1, wherein the auxiliary electrode is positioned in a shape-changing section of the medical device, and the method further comprises changing the shape of the shape-changing section to anchor the shape-changing section in a vessel.

10. The method of claim 9, wherein the target site comprises a wall of the vessel, and changing the shape of the shape-changing section positions the auxiliary electrode in contact with the target site.

11. The method of claim 9, further comprising using the medical device as a rail to advance a secondary device towards the target site.

12. The method of claim 9, wherein using the auxiliary electrode for diagnosis, mapping, or treatment at the target site comprises using the auxiliary electrode to perform a treatment at the target site, and then using the auxiliary electrode to confirm the treatment.

13. A method for carrying out a medical procedure, comprising:
advancing a medical device towards a puncture site in a patient's body and positioning a radiofrequency puncture electrode of the medical device adjacent the puncture site;
using an auxiliary electrode for diagnosis, mapping, or treatment of a target site;
delivering radiofrequency energy from the radiofrequency puncture electrode to create a puncture in the puncture site;
advancing the medical device through the puncture to position an auxiliary electrode of the medical device at a target site in the patient's body; and
using the auxiliary electrode for diagnosis, mapping, and/or treatment at the target site.

14. A medical device comprising:
an elongate member having a distal portion defining a distal end, and a proximal portion defining a proximal end, the elongate member comprising an inner mandrel and an outer liner;
a radiofrequency puncture electrode at the distal end, and a first electrical connector extending proximally from the radiofrequency puncture electrode towards the proximal end, for electrically connecting the radiofrequency puncture electrode to a radiofrequency generator;
at least a first auxiliary electrode in the distal portion, positioned proximally of the radiofrequency puncture electrode, and a second electrical connector extending proximally from the auxiliary electrode towards the proximal end, for electrically connecting the auxiliary electrode to a diagnostic system, a mapping system, and/or a treatment system.

15. The medical device of claim 14, wherein the first auxiliary electrode is one of a plurality of auxiliary electrodes that are longitudinally spaced apart in the distal portion.

16. The medical device of claim 15, wherein the distal portion comprises a shape-changing section, and the first auxiliary electrode is in the shape-changing section.

17. The medical device of claim 16, wherein the shape-changing section is changeable from a generally straight shape to a loop shape, a pigtail shape, a balloon shape, a basket shape, a J-shape, a semi-circular shape, or a combination thereof.

18. The medical device of claim 17, wherein the elongate member is relatively stiff proximal of the shape-changing section.

19. The medical device of claim 17, wherein the medical device is a guidewire and the elongate member has an outer diameter of between about 0.014 inches to about 0.060 inches.

20. The medical device of claim 17, wherein the medical device is a microcatheter and the elongate member has an outer diameter of between about 0.02 inches to about 0.079 inches.
